(19) United States
(12) Patent Application Publication
(10) Pub. No.: US 2021/0113851 A1
(43) Pub. Date: Apr. 22, 2021

(54) MEDICAL HANDPIECE AND MEDICAL LASER APPARATUS

(71) Applicant: LUTRONIC CORPORATION, Goyang (KR)

(72) Inventor: Hee Chul LEE, Goyang (KR)

(21) Appl. No.: 16/606,588

(22) PCT Filed: Apr. 10, 2018
(86) PCT No.: PCT/KR2018/004174

§ 371 Date: Oct. 18, 2019

(30) Foreign Application Priority Data
Apr. 19, 2017 (KR) 10-2017-0050314

(51) Int. Cl. A61N 5/06

(52) U.S. Cl. CPC A61N 5/06; A61N 2005/067

(57) ABSTRACT

A medical laser apparatus according to an embodiment of the present invention comprises: a laser oscillation unit for oscillating a laser; a beam width adjustment unit for adjusting the beam width of the laser oscillated from the laser oscillation unit; and a concentration unit for concentrating the laser of which the beam width has expanded by means of the beam width adjustment unit.

### TECHNICAL FIELD

The present invention relates to a medical handpiece and a medical laser apparatus, and more particularly, to a medical handpiece and a medical laser apparatus for use in a procedure using a laser.

### BACKGROUND ART

Recently, a treatment technique has been widely applied in such a manner that a human body is irradiated with a laser and a condition of tissue is changed by optical energy absorbed into human tissue.

A resonator is used to oscillate a laser. Resonators are divided into stable resonators and unstable resonators.

The unstable resonator has a larger single mode than the stable resonator due to its structural characteristics, but is disadvantageous in that, when focusing beams, a significant amount of beams are distributed outwardly due to diffraction and thereby side lobes are formed.

To remedy the disadvantage, an unstable resonator using a special mirror as an output mirror is used.

However, the laser oscillated from such an unstable resonator has a beam profile characteristic in that energy is outputted in the shape of tree growth rings.

### DISCLOSURE

### Technical Problem

An object of the present invention is to provide a medical handpiece and a medical laser apparatus capable of changing a profile of a beam oscillated from a laser source according to the preference or purpose of an operator.

Objects of the present invention are not limited to the above-mentioned objects. That is, other objects that are not mentioned may be obviously understood by those skilled in the art from the following description.

### Technical Solution

According to an embodiment of the present invention, a medical laser apparatus includes: a laser oscillation unit for oscillating a laser; a beam width adjustment unit for adjusting a beam width of the laser oscillated from the laser oscillation unit; and a concentration unit for concentrating the laser whose beam width has been expanded by the beam width adjustment unit.

The beam width adjustment unit may be movable between the laser oscillation unit and the concentration unit along an optical path of the laser oscillated from the laser oscillation unit.

As the beam width adjustment unit moves to be closer to the concentration unit, the laser emitted after passing through the concentration unit may have a larger beam width. As the beam width adjustment unit moves to be farther away from the concentration unit, the laser emitted after passing through the concentration unit may have a smaller beam width.

The beam width adjustment unit may include a concave lens.

The laser oscillation unit may include: a laser source for oscillating a laser of first beam profile; and a beam profile conversion unit for converting the laser of first beam profile oscillated from the laser source into a laser of second beam profile and transmitting the converted laser.

The beam profile conversion unit may produce the laser of second beam profile to have a more uniform luminous intensity distribution than the laser of first beam profile.

According to another embodiment of the present invention, a medical laser apparatus includes: a laser source for oscillating a laser of first beam profile; a beam profile conversion unit for converting the laser of first beam profile oscillated from the laser source into a laser of second beam profile and transmitting the converted laser; and a concentration unit for concentrating the laser of second beam profile.

The beam profile conversion unit may produce the laser of second beam profile to have a more uniform luminous intensity distribution than the laser of first beam profile.

The medical laser apparatus may further include a beam width adjustment unit provided between the beam profile conversion unit and the concentration unit to adjust a beam width of the laser of second beam profile having passed through the beam profile conversion unit.

The beam width adjustment unit may be movable between the beam profile conversion unit and the concentration unit along an optical path of the laser of second beam profile.

As the beam width adjustment unit moves to be closer to the concentration unit, the laser of second beam profile emitted after passing through the concentration unit may have a larger beam width. As the beam width adjustment unit moves to be farther away from the concentration unit, the laser of second beam profile emitted after passing through the concentration unit may have a smaller beam width.

According to another embodiment of the present invention, a medical handpiece connected to a main body to receive a laser oscillated from the main body and irradiating a subject with the laser for a procedure includes: a beam width adjustment unit for adjusting a beam width of the laser oscillated from the main body; and a concentration unit for concentrating the laser whose beam width has been expanded by the beam width adjustment unit.

The beam width adjustment unit may be movable so that a relative distance is variable between the beam width adjustment unit and the concentration unit.

As the beam width adjustment unit moves to be closer to the concentration unit, the laser emitted after passing through the concentration unit may have a larger beam width. As the beam width adjustment unit moves to be farther away from the concentration unit, the laser emitted after passing through the concentration unit may have a smaller beam width.

The medical handpiece may further include a beam profile conversion unit for converting the laser oscillated from the main body into a laser having another profile.

The laser converted to have another profile by the beam profile conversion unit may have a more uniform luminous intensity distribution than the laser of first beam profile.

According to another embodiment of the present invention, a medical handpiece connected to a main body to receive a laser oscillated from the main body and irradiating a subject with the laser for a procedure includes: a beam profile conversion unit for converting a laser of first beam profile oscillated from the main body into a laser of second beam profile and transmitting the converted laser; and a concentration unit for concentrating the laser of second beam profile.

The beam profile conversion unit may produce the laser of second beam profile to have a more uniform luminous intensity distribution than the laser of first beam profile.

The medical handpiece may further include a beam width adjustment unit provided between the beam profile conversion unit and the concentration unit to adjust a beam width of the laser of second beam profile having passed through the beam profile conversion unit.

The beam width adjustment unit may be movable between the beam profile conversion unit and the concentration unit along an optical path of the laser of second beam profile.

As the beam width adjustment unit moves to be closer to the concentration unit, the laser of second beam profile emitted after passing through the concentration unit may have a larger beam width. As the beam width adjustment unit moves to be farther away from the concentration unit, the laser of second beam profile emitted after passing through the concentration unit may have a smaller beam width.

Other specific details of the present invention are included in the detailed description and drawings.

### Advantageous Effects

According to embodiments of the present invention, the present invention has at least the following effect.

It is possible to change a profile of a beam oscillated from a laser source according to the preference or purpose of an operator.

The effect according to the present invention is not limited to the aforementioned effect, but more various effects are included in the present specification.

### DESCRIPTION OF DRAWINGS

FIG. 1 is a diagram schematically illustrating a medical laser apparatus according to a first embodiment of the present invention.

FIG. 2 is an image representing a profile of a laser before passing through a beam profile conversion unit.

FIG. 3 is an image representing a profile of the laser after passing through the beam profile conversion unit.

FIG. 4 is a diagram schematically illustrating a medical laser apparatus according to a second embodiment of the present invention.

FIG. 5 is a diagram schematically illustrating a medical laser apparatus according to a third embodiment of the present invention.

### BEST MODE

Advantages and features of the present invention and methods for accomplishment thereof will be apparent from embodiments of the present invention described in detail below together with the accompanying drawings. However, the present invention is not limited to the following embodiments but may be implemented in variously different forms. The embodiments are provided for those skilled in the art to fully understand the present invention and the scope of the present invention. The present invention is defined only by the scope of the appended claims. Throughout the specification, the same reference numerals and signs denote the same constituent elements.

Also, the embodiments described in the specification herein will be described with reference to cross-sectional views and/or schematic diagrams, which are ideal illustrations of the present invention. Therefore, the shapes of the illustrations may be modified according to, for example, manufacturing technology and/or permissible error. Also, each constituent element of the present invention in each of the drawings may be somewhat enlarged or reduced for convenience of description. Throughout the specification, the same reference numerals and signs denote the same constituent elements.

Hereinafter, the present invention will be described with reference to the accompanying drawings for describing a medical laser apparatus according to embodiments of the present invention.

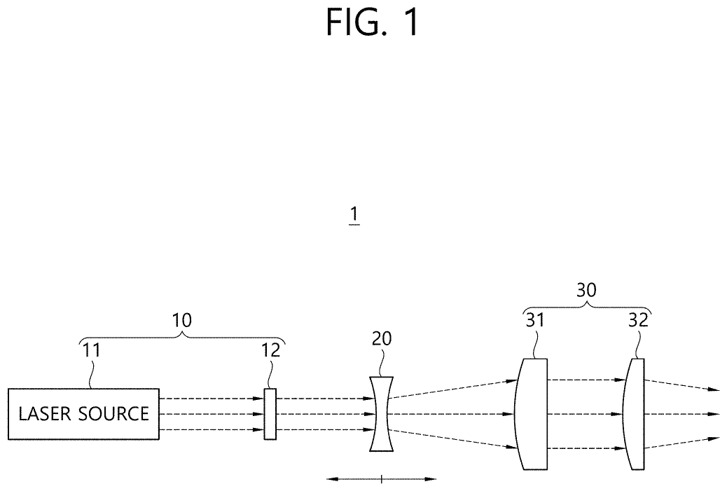

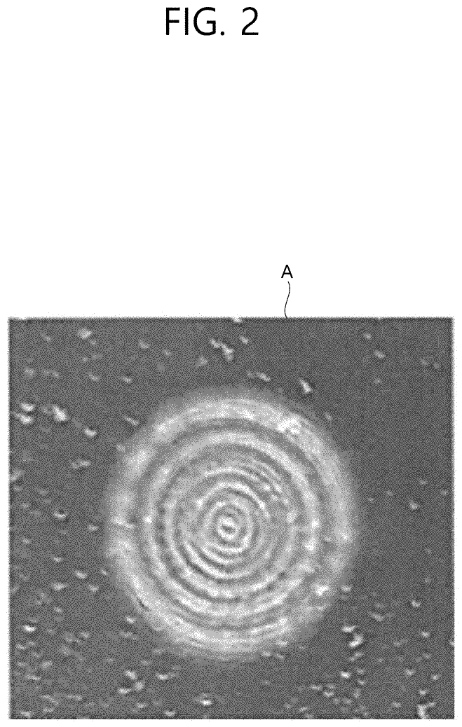

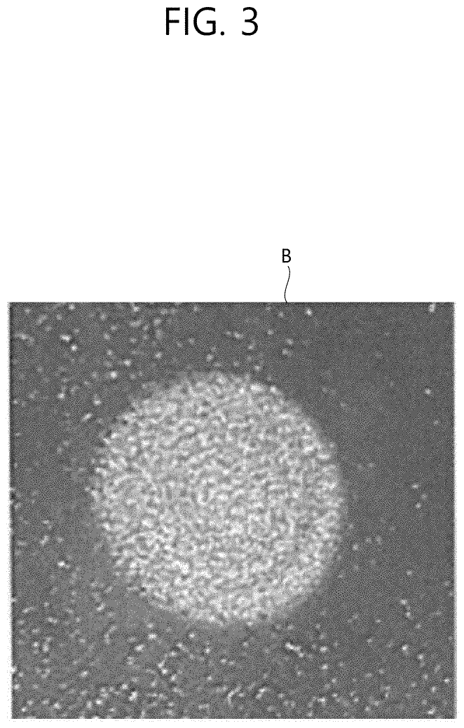

FIG. 1 is a diagram schematically illustrating a medical laser apparatus according to a first embodiment of the present invention, FIG. 2 is an image representing a profile of a laser before passing through a beam profile conversion unit, and FIG. 3 is an image representing a profile of the laser after passing through the beam profile conversion unit.

As illustrated in FIG. 1, the medical laser apparatus 1 according to the first embodiment of the present invention includes a laser oscillation unit 10, a beam width adjustment unit 20, and a concentration unit 30.

The laser oscillation unit 10 includes a laser source 11 and a beam profile conversion unit 12.

The laser source 11 oscillates a laser having a certain wavelength.

Although not shown, the laser source 11 may include a gain medium, a resonator, a pumping device, etc. to oscillate a laser of a specific wavelength.

The wavelength of the laser oscillated from the laser source 11 may vary depending on the gain medium. For example, when neodymium-doped yttrium aluminum garnet (Nd:YAG) is used as a gain medium, a laser having a wavelength of 1064 nm may be oscillated.

The beam profile conversion unit 12 converts a laser having a first beam profile, which has been oscillated from the laser source 11, into a laser having a second beam profile.

The beam profile of the laser refers to a cross-sectional shape perpendicular to an optical axis of the laser, a luminous intensity distribution characteristic of the laser, a diameter of the laser, and the like.

The laser of first beam profile oscillated from the laser source 11 is converted into the laser of second beam profile while passing through the beam profile conversion unit 12.

That is, the laser of second beam profile is changed from the laser of first beam profile in at least one characteristic of cross-sectional shape perpendicular to the optical axis, luminous intensity distribution, diameter, and the like.

For example, in the case where the cross-sectional shape of the laser of first beam profile is circular, the laser of first beam profile may be converted into the laser of second beam profile having a shape other than the circular shape, for example a tetragonal shape, while passing through the beam profile conversion unit 12.

Alternatively, the laser of second beam profile may have a circular cross-section with a larger diameter than the laser of first beam profile.

Alternatively, the beam profile conversion unit 12 may produce the laser of second beam profile by converting a luminous intensity distribution of the laser of first beam profile.

FIGS. 2 and 3 illustrate an example in which the luminous intensity distribution is converted by the beam profile conversion unit 12.

The image A shown in FIG. 2 is for a luminous intensity distribution of the laser of first beam profile. As shown in FIG. 2, the laser of first beam profile has a luminous intensity distribution in the shape of tree growth rings, like a laser outputted from an unstable resonator using a variable reflectivity mirror (VRM). That is, the luminous intensity is not uniform depending on the distance from the center of the laser.

The image B shown in FIG. 3 is for a luminous intensity distribution of the laser of second beam profile. As shown in FIG. 3, the laser of second beam profile has a uniform luminous intensity distribution like a laser outputted from a stable resonator, when compared to the laser of first beam profile. That is, the beam profile conversion unit 12 converts the laser of first beam profile into the laser of second beam profile to have a more uniform luminous intensity distribution.

Even though the laser oscillation unit 10 of the medical laser apparatus 1 according to the first embodiment of the present invention uses an unstable resonator for the laser source 11, the laser oscillation unit 10 is capable of oscillating a laser having a luminous intensity distribution similar to that of a laser outputted from a stable resonator by using the beam profile conversion unit 12.

A diffuser may be used as the beam profile conversion unit 12.

Meanwhile, as illustrated in FIG. 1, the beam width adjustment unit 20 is provided between the beam profile conversion unit 12 and the concentration unit 30.

The beam width adjustment unit 20 expands a beam width of the laser of second beam profile having passed through the beam profile conversion unit 12. To do so, the beam width adjustment unit 20 may include at least one lens, and the lens constituting the beam width adjustment unit 20 may include a concave lens.

The beam width adjustment unit 20 is movable between the beam profile conversion unit 12 and the concentration unit 30 along an optical path of the laser of second beam profile. A relative distance between the beam width adjustment unit 20 and the concentration unit 30 varies as the beam width adjustment unit 20 moves.

As illustrated in FIG. 1, the concentration unit 30 concentrates the laser whose beam width has been expanded while passing through the beam width adjustment unit 20.

The concentration unit 30 may include a plurality of lenses 31 and 32 to concentrate the laser having passed through the beam width adjustment unit 20. As an example of the concentration unit 30, it is illustrated in FIG. 1 that the concentration unit 30 includes a collimating lens 31 and a convex lens 32, but the concentration unit 30 is not limited thereto. The concentration unit 30 may include at least one of the collimating lens 31, the convex lens 32, and an aspherical lens.

The collimating lens 31 refracts the laser whose beam width has been expanded while passing through the beam width adjustment unit 20 such that the beams are approximately parallel with respect to the optical axis.

The convex lens 32 refracts and concentrates the laser whose beams have become parallel to each other while passing through the collimating lens 31.

As the beams incident on the concentration unit 30 have a larger diameter, the concentration unit 30 concentrates the laser to be irradiated with a smaller beam width at a point spaced apart from the concentration unit 30 at a predetermined distance.

Therefore, as the beam width adjustment unit 20 is closer to the concentration unit 30 by moving toward the concentration unit 30, the diameter of the beam incident on the concentration unit 30 is smaller, and accordingly, the beam width of the laser irradiated at a point spaced apart from the concentration unit 30 at a predetermined distance is larger.

Reversely, as the beam width adjustment unit 20 is farther away from the concentration unit 30 by moving toward the beam width adjustment unit 20, the diameter of the beam incident on the concentration unit 30 is larger, and accordingly, the beam width of the laser irradiated at a point spaced apart from the concentration unit 30 at a predetermined distance is smaller.

Therefore, an operator may adjust a beam width and/or intensity of a laser irradiated to a subject by moving the beam width adjustment unit 20.

As described above, the medical laser apparatus 1 according to an embodiment of the present invention includes the beam profile conversion unit 12, thereby making it possible to use a laser that is converted to have a new beam profile including a cross-sectional shape, a luminous intensity distribution, a diameter, and the like, without using the original beam profile of the laser emitted from the laser source 11.

In addition, it is possible to convert the beam profile of the laser emitted from the laser source using the unstable resonator to be similar to the beam profile of the laser emitted from the laser source using the stable resonator.

Therefore, in the medical field where the laser source 11 is restrictive, it is possible to emit lasers of various beam profiles and change the beam profile according to the operator preference or purpose.

In addition, the beam width adjustment unit 20 and the concentration unit 30 are configured in such a manner that a relative distance therebetween may be adjusted, thereby making it possible to adjust a beam width and/or intensive of a laser irradiated to the subject.

Hereinafter, a medical laser apparatus according to another embodiment will be described.

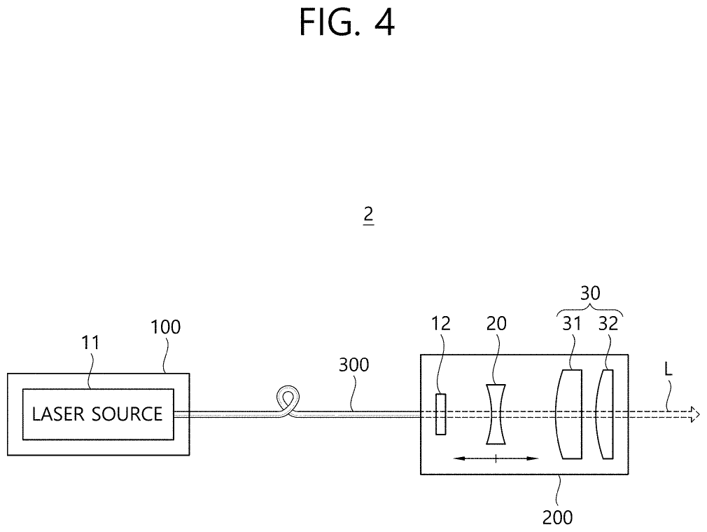

FIG. 4 is a diagram schematically illustrating a medical laser apparatus according to a second embodiment of the present invention. For convenience of description, the elements similar to those of the first embodiment are denoted by the same reference numerals and signs, and the description of the elements that is common to the first embodiment will be omitted.

The medical laser apparatus 2 according to the second embodiment of the present invention includes a main body 100 and a handpiece 200.

The handpiece 200, which is a tool to be held by an operator for performing a procedure, is connected through a flexible optical cable 300, and receives a laser L emitted from the main body 100 and irradiates a subject with the laser for the procedure.

As illustrated in FIG. 4, in the medical laser apparatus 2 according to the second embodiment of the present invention, a laser source 11 is provided in the main body 100, and a beam profile conversion unit 12, a beam width adjustment unit 20, and a concentration unit 30 are provided in the handpiece 200.

Therefore, a laser of a first beam profile oscillated from the laser source 11 is incident on the handpiece 200 along the optical cable 300, and converted into a laser of second beam profile while passing through the beam profile conversion unit 12 in the handpiece 200. Thereafter, the converted laser is emitted through the beam width adjustment unit 20 and the concentration unit 30.

The beam width adjustment unit 20 is provided in the handpiece 200 in such a manner as to be movable between the beam profile conversion unit 12 and the concentration unit 30. In order to enable an operator to adjust a location of the beam width adjustment unit 20, a manipulation unit (now shown) for shifting the location of the beam width adjustment unit 20 may be provided on an external side of the handpiece 200. The operator may adjust a beam width and/or intensity of the laser irradiated to a subject by controlling the location of the beam width adjustment unit 20 through the manipulation unit.

In addition, the handpiece 200 of the medical laser apparatus 2 according to the second embodiment may be configured in such a manner that the beam profile conversion unit 12 is replaceable. Therefore, it is possible to emit a laser L having a beam profile suitable for the operator's preference or purpose based on the beam profile conversion unit 12 to be replaced.

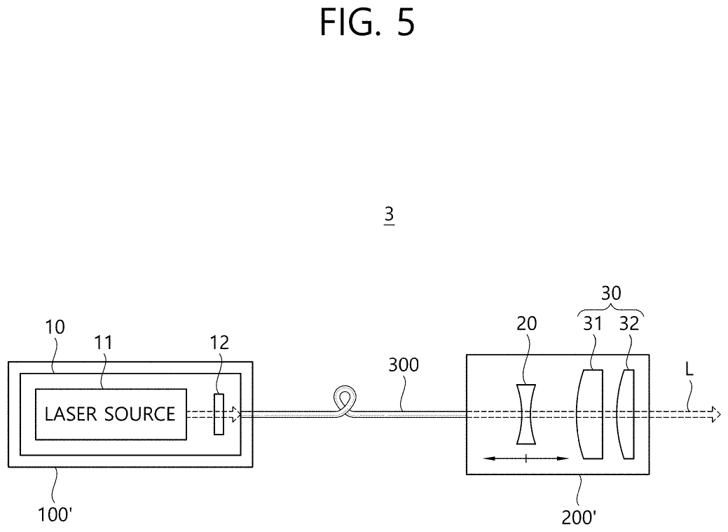

FIG. 5 is a diagram schematically illustrating a medical laser apparatus according to a third embodiment of the present invention. For convenience of description, the elements similar to those of the second embodiment are denoted by the same reference numerals and signs, and the description of the elements that is common to the second embodiment will be omitted.

As illustrated in FIG. 5, in the medical laser apparatus 3 according to the third embodiment of the present invention, a laser oscillation unit 10 is provided in a main body 100′, and a beam width adjustment unit 20 and a concentration unit 30 are provided in a handpiece 200′.

Therefore, a laser of first beam profile oscillated from a laser source 11 is converted into a laser of second beam profile while passing through the beam profile conversion unit 12 in the main body 100′. Thereafter, the converted laser is incident on the handpiece 200′ through an optical cable 30.

The handpiece 200′ according to the third embodiment is also configured such that the beam width adjustment unit 20 is movable in the handpiece 200′, but the beam profile conversion unit 12 is located in the main body 100′. Therefore, the beam width adjustment unit 20 is provided such that its relative distance is variable with respect to the concentration unit 30.

In order to enable an operator to adjust a location of the beam width adjustment unit 20, the handpiece 200′ according to the third embodiment may also be provided with a manipulation unit (now shown) for shifting the location of the beam width adjustment unit 20 on an external side of the handpiece 200′. The operator may adjust a beam width and/or intensity of the laser irradiated to a subject by controlling the location of the beam width adjustment unit 20 through the manipulation unit.

It is to be understood by those skilled in the art that the present invention may be embodied in another specific form without changing the technical spirit or essential features thereof. It is therefore to be understood that the above-described embodiments are illustrative in all aspects and not restrictive. The scope of the present invention is defined by the following claims rather than the above detailed description, and all changes or modifications derived from the meaning and scope of the claims and their equivalents should be construed as being included in the scope of the present invention.

### Mode for Invention

A medical laser apparatus according to an embodiment of the present invention includes a laser oscillation unit for oscillating a laser, a beam width adjustment unit for adjusting a beam width of the laser oscillated from the laser oscillation unit, and a concentration unit for concentrating the laser whose beam width has been expanded by the beam width adjustment unit.

A medical handpiece according to an embodiment of the present invention is a medical handpiece connected to a main body to receive a laser oscillated from the main body and irradiating a subject with the laser for a procedure, and includes a beam width adjustment unit for adjusting a beam width of the laser oscillated from the main body, and a concentration unit for concentrating the laser whose beam width has been expanded by the beam width adjustment unit.

A medical handpiece according to an embodiment of the present invention is a medical handpiece connected to a main body to receive a laser oscillated from the main body and irradiating a subject with the laser beam for a procedure, and includes a beam profile conversion unit for converting a laser of first beam profile oscillated from the main body into a laser of second beam profile and transmitting the converted laser, and a concentration unit for concentrating the laser of second beam profile.

1. A medical laser apparatus, comprising:
a laser oscillation unit for oscillating a laser;
a beam width adjustment unit for adjusting a beam width of the laser oscillated from the laser oscillation unit; and
a concentration unit for concentrating the laser whose beam width has been expanded by the beam width adjustment unit.

2. The medical laser apparatus of claim 1, wherein the beam width adjustment unit is movable between the laser oscillation unit and the concentration unit along an optical path of the laser oscillated from the laser oscillation unit.

3. The medical laser apparatus of claim 2, wherein as the beam width adjustment unit moves to be closer to the concentration unit, the laser emitted after passing through the concentration unit has a larger beam width, and
as the beam width adjustment unit moves to be farther away from the concentration unit, the laser emitted after passing through the concentration unit has a smaller beam width.

4. The medical laser apparatus of claim 1, wherein the beam width adjustment unit includes a concave lens.

5. The medical laser apparatus of claim 1, wherein the laser oscillation unit includes:
a laser source for oscillating a laser of first beam profile; and
a beam profile conversion unit for converting the laser of first beam profile oscillated from the laser source into a laser of second beam profile and transmitting the converted laser.

6. The medical laser apparatus of claim 5, wherein the beam profile conversion unit produces the laser of second beam profile to have a more uniform luminous intensity distribution than the laser of first beam profile.

7. A medical laser apparatus, comprising:
a laser source for oscillating a laser of first beam profile;
a beam profile conversion unit for converting the laser of first beam profile oscillated from the laser source into a laser of second beam profile and transmitting the converted laser; and
a concentration unit for concentrating the laser of second beam profile.

8. The medical laser apparatus of claim 7, wherein the beam profile conversion unit produces the laser of second beam profile to have a more uniform luminous intensity distribution than the laser of first beam profile.

9. The medical laser apparatus of claim 7, further comprising a beam width adjustment unit provided between the beam profile conversion unit and the concentration unit to adjust a beam width of the laser of second beam profile having passed through the beam profile conversion unit.

10. The medical laser apparatus of claim 9, wherein the beam width adjustment unit is movable between the beam profile conversion unit and the concentration unit along an optical path of the laser of second beam profile.

11. The medical laser apparatus of claim 10, wherein as the beam width adjustment unit moves to be closer to the concentration unit, the laser of second beam profile emitted after passing through the concentration unit has a larger beam width, and
as the beam width adjustment unit moves to be farther away from the concentration unit, the laser of second beam profile emitted after passing through the concentration unit has a smaller beam width.

12-16. (canceled)

17. A medical handpiece connected to a main body to receive a laser oscillated from the main body and irradiating a subject with the laser for a procedure, comprising:
a beam profile conversion unit for converting a laser of first beam profile oscillated from the main body into a laser of second beam profile and transmitting the converted laser; and
a concentration unit for concentrating the laser of second beam profile.

18. The medical handpiece of claim 17, wherein the beam profile conversion unit produces the laser of second beam profile to have a more uniform luminous intensity distribution than the laser of first beam profile.

19. The medical handpiece of claim 17, further comprising a beam width adjustment unit provided between the beam profile conversion unit and the concentration unit to adjust a beam width of the laser of second beam profile having passed through the beam profile conversion unit.

20. The medical handpiece of claim 19, wherein the beam width adjustment unit is movable between the beam profile conversion unit and the concentration unit along an optical path of the laser of second beam profile.

21. The medical handpiece of claim 20, wherein as the beam width adjustment unit moves to be closer to the concentration unit, the laser of second beam profile emitted after passing through the concentration unit has a larger beam width, and
as the beam width adjustment unit moves to be farther away from the concentration unit, the laser of second beam profile emitted after passing through the concentration unit has a smaller beam width.
